(19) United States
(12) Patent Application Publication
(10) Pub. No.: US 2008/0071280 A1
(43) Pub. Date: Mar. 20, 2008

(54) System and Method for Insertion of an Interspinous Process Implant that is Rotatable in Order to Retain the Implant Relative to the Spinous Processes

(75) Inventor: Charles J. Winslow, Walnut Creek, CA (US)

Correspondence Address: COATS & BENNETT/MEDTRONICS, 1400 CRESCENT GREEN, SUITE 300, CARY, NC, 27518, US

(73) Assignee: ST. FRANCIS MEDICAL TECHNOLOGIES, INC., Sunnyvale, CA (US)

(21) Appl. No.: 11/874,131

(22) Filed: Oct. 17, 2007

Related U.S. Application Data
(63) Continuation of application No. 11/003,555, filed on Dec. 3, 2004.
(60) Provisional application No. 60/565,910, filed on Apr. 28, 2004.

(51) Int. Cl. A61B 17/70; A61F 2/44

(52) U.S. Cl. 606/90; 606/61; 623/17.11

(57) ABSTRACT

The present invention is directed to a device that is implanted between the spinous processes of adjacent vertebrae of the spine and used for relieving pain associated with the vertebrae and surrounding tissues and structures by maintaining and/or adding distraction between adjacent vertebrae. The present invention includes a tissue expander adapted to move from a first insertion position, for ease of implantation between spinous processes, to a second retention position that prevents displacement of the implant.

### CROSS-REFERENCES TO RELATED APPLICATIONS

This U.S. patent application is a continuation of and claims priority from U.S. application Ser. No. 11/003,555, filed on Dec. 3, 2004, which claims the benefit of under 35 U.S.C. § 109(e) of U.S. Provisional Patent Application No. 60/565,910, as filed on Apr. 28, 2004, the disclosures of which are incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

This invention relates to an interspinous process implant and method for implantation.

The spinal column is a biomechanical structure composed primarily of ligaments, muscles, vertebrae and intervertebral disks. The biomechanical functions of the spine include: 1) support of the body, which involves the transfer of the weight and the bending movements of the head, trunk and arms to the pelvis and legs; (2) complex physiological motion between these parts; and (3) protection of the spinal cord and nerve roots.

As the present society ages, it is anticipated that there will be an increase in adverse spinal conditions which are characteristic of older people. By way of example, with aging comes an increase in spinal stenosis (including, but not limited to, central canal and lateral stenosis), and facet anthropathy. Spinal stenosis typically results from the thickening of the bones that make up the spinal column and is characterized by a reduction in the available space for the passage of blood vessels and nerves.

Pain associated with such stenosis can be relieved by medication and/or surgery. It is desirable to eliminate the need for major surgery for all individuals, and in particular, for the elderly.

Accordingly, a need exists to develop spine implants that alleviate pain caused by spinal stenosis and other such conditions caused by damage to, or degeneration of, the spine. Such implants would distract, or increase the space between, the vertebrae to increase the foramina 1 area and reduce pressure on the nerves and blood vessels of the spine.

Further, a need exists for an implant that minimizes further trauma to the spine, and obviates the need for invasive methods of surgical implantation. Additionally, a need exists to address adverse spinal conditions that are exacerbated by spinal extension.

### BRIEF SUMMARY OF THE INVENTION

### BRIEF DESCRIPTION OF THE DRAWINGS

FIG. 1 depicts a side view of an embodiment of the implant of the invention, in a first insertion position.

FIG. 2 is a top-down view of the embodiment of the implant of the invention depicted in FIG. 1 in the first insertion position.

FIG. 3 is a side view of the embodiment of the implant of the invention depicted in FIG. 1 in a second retention position.

FIG. 4 illustrates a top-down view of an embodiment of the implant of the invention, the implant positioned under a spinous process of the spine with the tissue expander in the second retention position.

FIG. 5 depicts a side view of an alternative embodiment of the implant of the invention in a first insertion position.

FIG. 6 depicts a side view of the alternative embodiment of the implant of the invention illustrated in FIG. 5, in a second retention position.

FIG. 7 depicts a top view of yet another embodiment of the implant of the invention, in a first insertion position.

FIG. 8 depicts a side view of the embodiment shown in FIG. 9 of the implant of the invention, in a second retention position.

FIG. 9 depicts a top view of the embodiment of FIG. 7 in a deployed position between spinous processes.

FIG. 10 depicts a flow diagram of a method of insertion of an implant of the invention.

### DETAILED DESCRIPTION OF THE INVENTION

Embodiments of the present invention relate to an interspinous process implant including a first wing for implant retention after placement, a spacer for maintaining and/or causing additional distraction, and a tissue expander for converting from a first position for insertion to a second position for retention of the implant after placement between adjacent spinous processes. In the second position, the tissue expander acts like a second wing to prevent displacement of the implant. The disclosed invention further claims a method for lateral insertion of the disclosed implant of the invention.

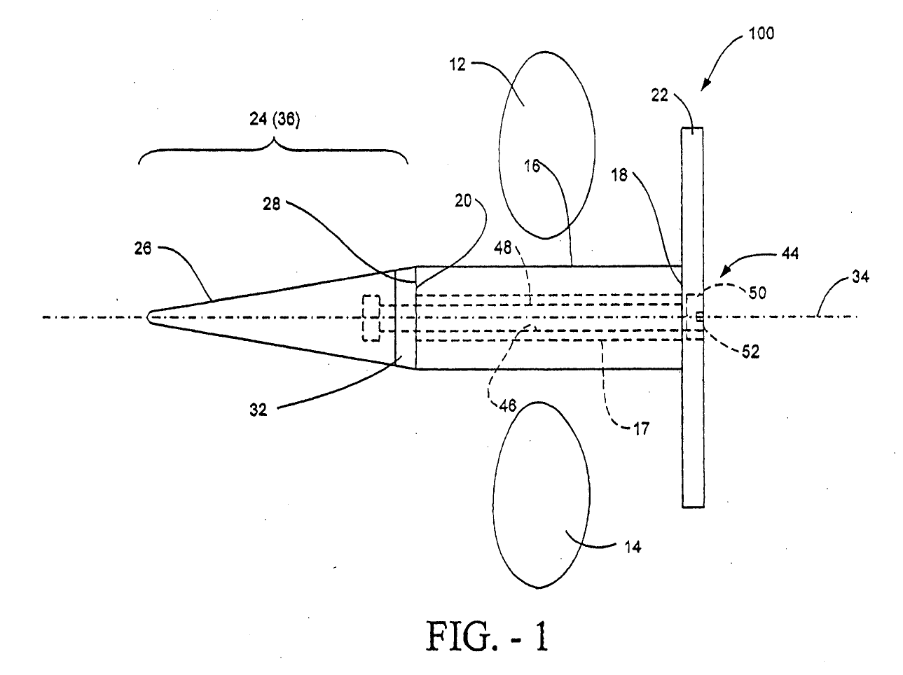

FIG. 1 shows a side view of an embodiment of an implant 100 of the disclosed invention. The implant 100 comprises a spacer 16 that maintains the distraction of the spinous processes of adjacent vertebrae, once the spacer 16 is positioned between the spinous processes 12, 14. The spacer 16 can have various shapes including, by way of example only, a cylindrical shape, an elliptical shape, or tear-drop shape when viewed in cross-section substantially perpendicular to a longitudinal or elongated axis 34 of the spacer 16. The longitudinal axis 34 is oriented from the left lateral to right lateral spine, when the implant 100 is positioned in the spine.

The spacer 16 has a first or proximal end 18 and a second or distal end 20. At the first end 18, the spacer 16 is connected with a first wing 22. The first wing 22 functions as a first retaining unit. That is, the first wing 22 prevents displacement of the implant 100 once the implant 100 is positioned in the spine, with the spacer 16 between adjacent spinous processes. From the first wing 22 extends a shaft 17 upon which the rotatable spacer 16 is rotatably mounted, so that the spacer 16 can rotate independently from the first wing 22 for positioning of both elements of the implant 100. Alternatively, the first wing 22 can be fixedly connected with, or integral to the spacer 16.

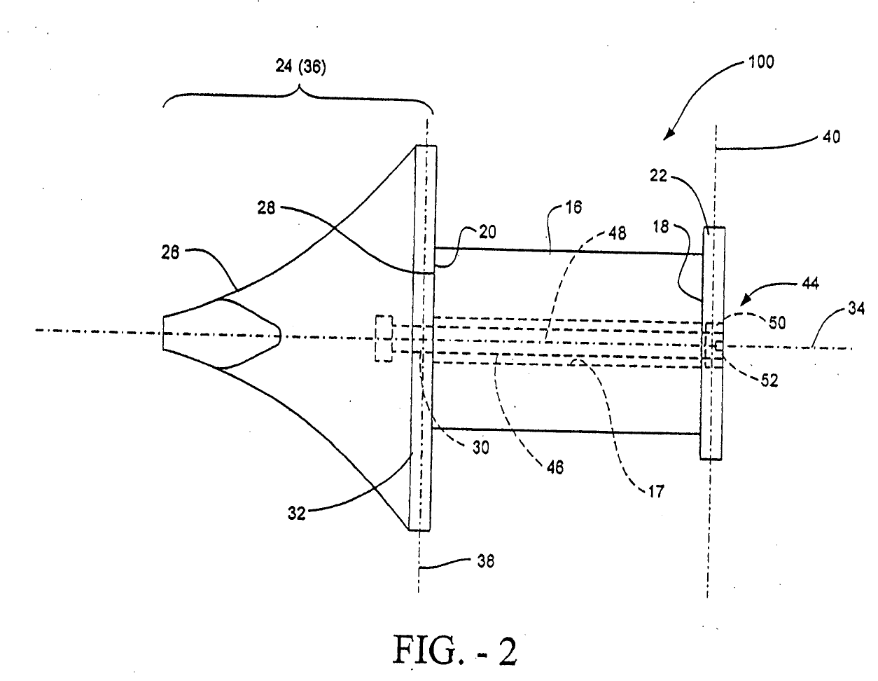

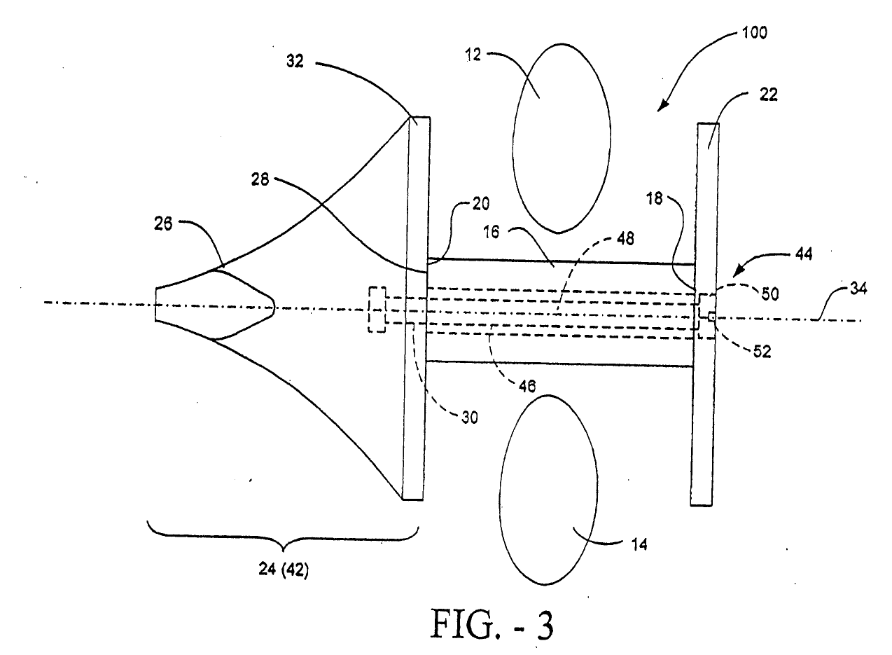

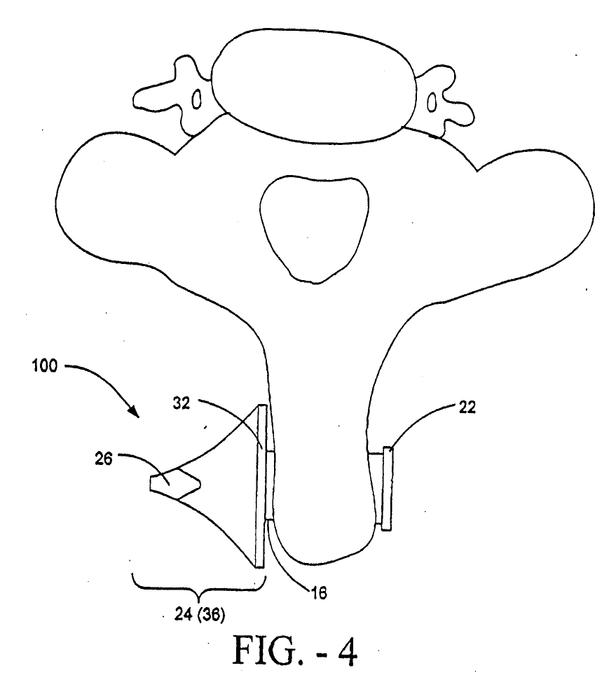

The second end 20 of the spacer 16 is located adjacent to a tissue expander 24. The tissue expander 24 has a wedge-shaped first end 26 that is distal to the spacer 16, and a second end 28 that is adjacent the spacer 16. As discussed below, the tissue expander 24 can be rotated about the axis 34. When the tissue expander 24 is rotated about the rotation axis 34, it is converted from a first insertion position 36 (shown in FIGS. 1 and 2) to a second retention position 42 (shown in FIGS. 3 and 4).

In FIGS. 1 and 2, the tissue expander 24 is in the first insertion position 36, where FIG. 1 is a side view of an embodiment of the implant of the invention, and FIG. 2 is a top-down view of an embodiment of the implant 100 of the invention, in the same first insertion position 36 as the implant 100 depicted in FIG. 1. The first end 26 of the tissue expander 24 is wedge-shaped in the direction of insertion, where the implant 100 is inserted laterally. That is, where the implant 100 is to be inserted laterally, the wedge-shaped first end 26 is narrowest at the point of insertion and broadens along the length of the tissue expander 24 toward the second end 28 of the tissue expander 24 that is located adjacent to the second end 20 of the spacer 16. The wedge shape of the tissue expander 24 facilitates insertion of the implant 100 by initiating distraction, if no other method is used during implantation, or by adding to or maintaining distraction created by another source, if any other such source is employed.

FIG. 2 shows a top-down view of the implant 100, with the tissue expander 24 in the first insertion position 36. The elongated base element 32 of the tissue expander 24 in the first insertion position 36, where insertion is from a lateral direction, is oriented in an anterior-posterior direction relative to a patient. In contrast, as discussed in greater detail below, the elongated base element 32 of the tissue expander 24 in the second retention position 42 is oriented substantially perpendicular to the first insertion position 36, in a direction that is substantially perpendicular to the anterior-posterior direction relative to the patient. In this embodiment, the elongated base element 32 in the first insertion position 36 can further be described as substantially perpendicular to the orientation of the first wing 22, the first wing 22 being at about a 90″ angle from the anterior-posterior direction relative to the patient, substantially parallel to the axial plane of the patient.

FIG. 3 depicts a side view of the implant 100 with the tissue expander 24 rotated to the second retention position 42. The tissue expander 24 in the second retention position 42 can prevent displacement of the implant 100 after insertion in the spine of a patient. The tissue expander 24, including the wedge-shaped first end 26, and the elongated element 32, can rotate from the first insertion position 36 (FIG. 1) adapted to facilitate insertion, to the second retention position 42 (FIG. 3) after insertion and positioning of the implant 100.

In one embodiment, the tissue expander 24 rotates about 90″ to be reconfigured into the second retention position 42, which alters the orientation of the wedge-shaped first end 26 of the tissue expander 24 and the elongated base element 32. In the second retention position 42, the tissue expander 24 is rotated about 90″ so that the elongated element 32 (FIG. 3) is substantially perpendicular to the anterior-to-posterior direction of a patient. In other words, instead of being oriented with the axis 38 of the elongated element 32 from anterior to posterior (FIGS. 1 (side view) and 2 (top-down view)), the elongated base element 32 is rotated about the elongated axis 34 of the spacer 16, so that the elongated base element 32 is oriented generally parallel to the first wing 22 (FIG. 3). It will be understood by those of ordinary slull in the art that the shift need not be 90″ and could be by way of example of 45″ or 60″.

FIG. 4 depicts the embodiment of the implant 100, positioned between adjacent spinous processes, upon initial insertion and in the configuration of FIGS. 1 and 2.

The tissue expander 24 can be locked into the second retention position 42, as depicted in FIGS. 1-3. In this embodiment, the shaft 17 has a bore 46 extending completely therethrough, which bore 46 has the same longitudinal axis 34 as does shaft 17. Positioned in bore 46 is a shaft 48 which can rotate in bore 46, and which is connected to tissue expander 24. Shaft 48 includes a head 50 which has a slot 52 that can accept a rotation tool, such as a screwdriver. Rotation of the shaft 48 causes the tissue expander 24 to rotate. Thus once the implant 100 is positioned between spinous processes, a screwdriver can be used to rotate the tissue expander 24 from the insertion position as seen in FIGS. 1, and 2 to the retention position shown in FIG. 3. In a preferred embodiment the shaft 48 can rotate the tissue expander 24 about 90″. Alternatively, different amounts of rotation can be accomplished. Although the patient's tissue will hold the tissue expander 24 in the rotated position, if desired, a mechanism can be included to fix the shaft 48 in the rotated position. Such mechanism can include a detent extending from head 50 which can lock into a recess in the first wing 22 as the shaft 48 is rotated. Another mechanism can include ridges extending from the head 50 of the shaft 48 which can lock into recesses in the first wing 22. One of ordinary skill in the art can appreciate that other lock-and-key mechanisms, or other mechanism that allows rotation and locking into the desired second retention position 42, can also be employed to secure the second retention position 42 for implant 100.

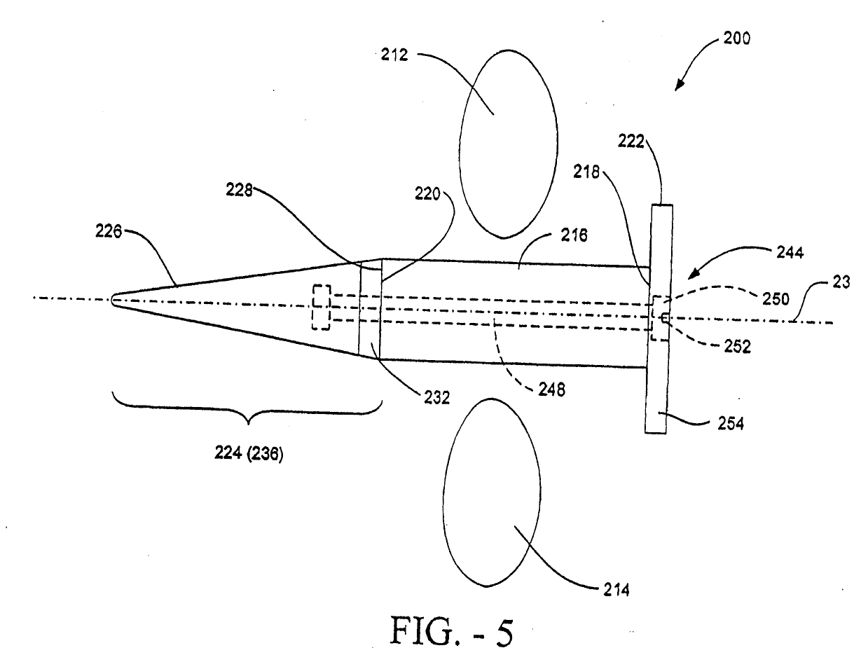

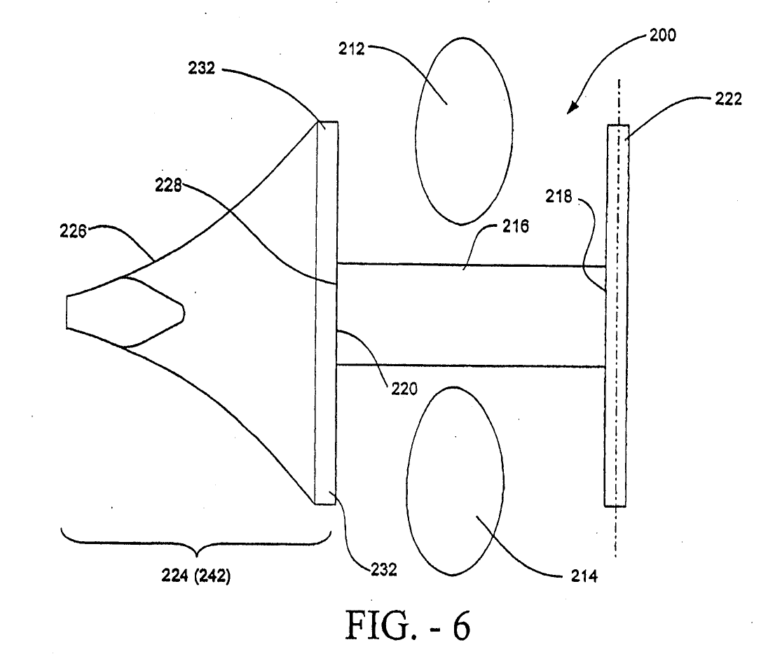

FIGS. 5 and 6 depict an alternative embodiment of the implant 200 of the disclosed invention. In this embodiment, both the first wing 222 and the tissue expander 224, are secured to shaft 248 and can rotate together, from a first insertion position 236 (FIG. 5) to a second retention position 242, (FIG. 6) once the implant 200 is positioned between the adjacent spinous processes. In the first insertion position 236, the first wing 222 and an elongated base element 232 of the tissue expander 224 are oriented for ease of insertion in an anterior-to-posterior direction of the patient. In the second retention position 242, for second wing 226 (FIG. 6) the elongated base element 232 of the tissue expander 224 and the first wing 222 are oriented about 90″ from the first insertion position 236, or in other words, substantially perpendicular to the anterior-to-posterior direction of the first insertion position 236.

The implant 200 has a tissue expander 224 having a wedge-shaped distal end 226 and a proximal end 228 that is located adjacent to rotatable spacer 216 at a second distal end 220 of a rotatable spacer 216. A first wing 222 is located adjacent to a proximal first end 218 of spacer 216. Focusing first on the tissue expander 224, the wedge-shaped distal end 226 is oriented in the first insertion position 236 to accommodate insertion between spinous processes 212,214, with the flat distal part of the wedge 226 oriented in an anterior-to-posterior direction in a patient. Also in the first insertion position 236, the elongated base element 232 of the tissue expander 224, located adjacent to the spacer 216, is oriented so that it is elongated in a direction that is anterior-to-posterior when the implant 200 is implanted laterally in a patient.

With respect to the first wing 222, when the tissue expander 224 is oriented in the first insertion position 236, the first wing 222 is oriented, like the tissue expander 224, in an anterior-to-posterior direction relative to a patient. As with the tissue expander 226 rotation of the shaft 248 causes the first wing 222 to rotate so that is about perpendicular to an anterior-posterior direction.

In this embodiment, as indicated above, the first wing 222 and the tissue expander 224 are joined together by the shaft 248 which has a longitudinal axis 234. The spacer 216 can rotate upon shaft 248. Shaft 248 includes a head 250 which has a slot 252 that can accept a rotation tool such as a screwdriver. Rotation of the shaft 248 causes the first wing 222 as well as the tissue expander 224 to rotate. Thus, once the implant 200 is positioned between spinous processes, a screwdriver can be used to rotate the tissue expander 224 and the first wing 222 from the insertion position 236 as seen in FIG. 5 to the retention position 242 shown in FIG. 6. In a preferred embodiment, the shaft 248 can rotate the first wing 222 as well as the tissue expander 224 about 90″. Alternatively, different amounts of rotation can be accomplished.

Although the patient's tissue will hold the first wing 222 and the tissue expander 224 in the rotated position, if desired, a mechanism can be included to fix the shaft 248 in the rotated position. Such mechanism can include a detent extending from the shaft 248 which can lock into a recess in the spacer 216. Accordingly, in the second retention position 242, both the first wing 222 and the tissue expander 224 are rotated about 90″ and can be locked into place.

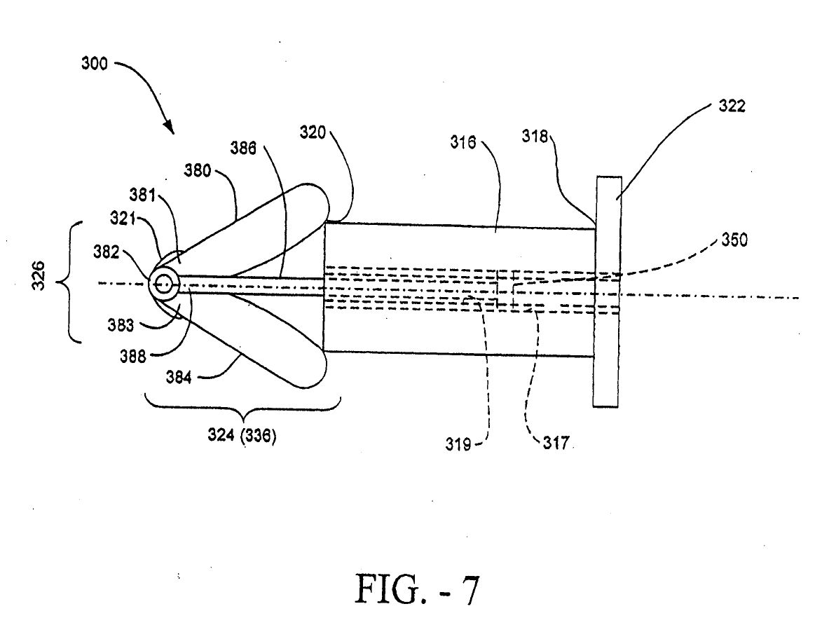

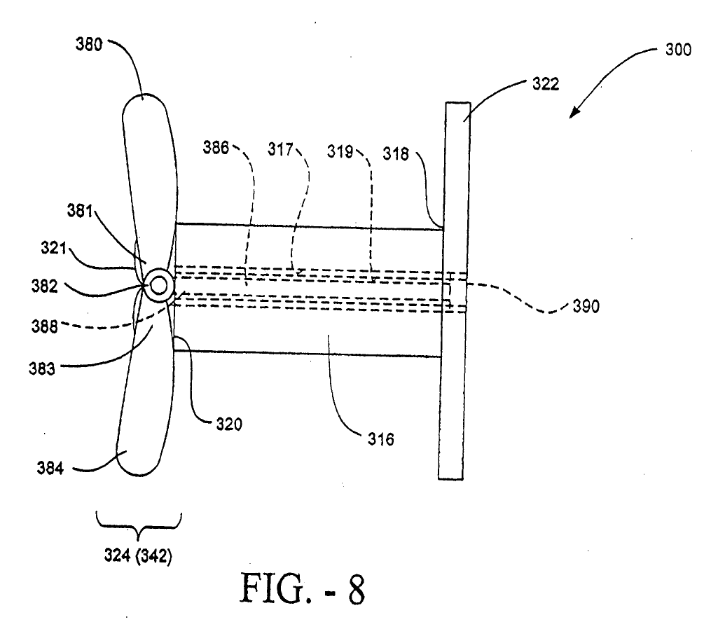

FIGS. 7 and 8 depict yet another embodiment 300 of the implant of the invention. In this embodiment 300, the tissue expander 324 has a first insertion position 336 (FIG. 7 being a view looking down on the spinal column), and a second retention position 342 (FIG. 8 being a view looking from posterior to anterior into the spinal column). The tissue expander 324 converts between the first insertion position 336 and the second retention 342 position through a pivoting motion, that may also include a rotation motion.

In this embodiment 300, the first wing 322 is positioned adjacent to a spacer 316 at a first end 318 of the spacer. As above with the implants 100 and 200, the spacer 316 is rotatably mounted over a hollow spacer-mounting shaft 317 extending from the first wing 322. The spacer 316 can be cylindrical, or it can have other shapes, including but not limited to elliptical or tear-drop shape in cross-section.

The tissue expander 324 of implant 300 comprises an upper segment 380 that is pivotally connected via a pivoting joint 382, or other pivoting means, with a lower segment 384. That is, a second end 381 of the upper segment 380 meets a second end 383 of the lower segment 384 via the pivoting joint 382. A coiled spring 321 is provided around pivoting joint 382 and biees both the upper segment 380 and the lower segment 384 of the tissue expander 324 against the spacer 316. The pivoting joint 382 is connected with a first end 388 of a rod 386. Rod 386 is slidably disposed in a bore 319 which runs the entire length of the spacer 316, and is located within hollow spacer-mounting shaft 317.

The pivoting joint 382 and the rod 386 provide the mechanism whereby the tissue expander 324 is converted from the first insertion position 336 to the second retention position 342. In the first insertion position 336, depicted in FIG. 7, the first end 388 of the rod 386 extends outside the spacer 316. The pivoting joint 382, functionally connected with the first end 388 of the rod 386, is not in contact with the second end 320 of the spacer 316, but instead is separated by a segment of the rod 386 from the second end 320 of the spacer 316. The upper segment 380 of the tissue expander 324 and the lower segment 384 of the tissue expander 324 meet at the pivoting joint 382 to form a wedge-shaped first end 326 of the tissue expander 324 that is not in contact with the second end 320 of the spacer 316 when the tissue expander 324 is in the first insertion position 336. In one embodiment, wedge-shaped first end 326 of the tissue expander 324 can be wedge-shaped in the lateral direction of insertion, i.e., perpendicular to an anterior-to-posterior direction of a patient. The wedge-shaped first end 326 is useful for inserting the implant 300 between adjacent spinous processes.

Rod 386 includes a head 350 at the end of the rod 386 distal from the tissue expander 324. The head 350 has a slot 352 that can accept a tool adapted to be used to rotate and pull the rod 386 through a bore 319 of shaft 317 toward the first wing, causing the upper segment 380 and lower segment 384 of the tissue expander 324 to become aligned, such that the tissue expander 324 is no longer wedge-shaped in the first insertion position 336 (FIG. 7). Instead, the tissue expander 324 adopts the form of a second wing (FIG. 8). In this embodiment, the tissue expander 324 is wedge-shaped in the direction of lateral insertion in the first insertion position 336, and the tissue expander in the second retention position is oriented substantially vertical, or substantially perpendicular to the anterior-to-posterior direction of the patient.

In contrast, in another embodiment, the tissue expander 324 in the first insertion position 336 is not wedge-shaped in a top view during lateral insertion, as discussed above. Instead, the wedge-shape of the tissue expander 324 in the first insertion position 336 is wedge-shaped looking into the spine from a posterior to anterior direction. As such, merely pulling without rotating rod 386 causes upper segment 380 of the tissue expander 324 and the lower segment 384 of the tissue expander 324 to pivot about the pivoting joint 382, as above. Thus without rotating the tissue expander 324, the tissue expander 324 after reconfiguration will be oriented as depicted in FIG. 8.

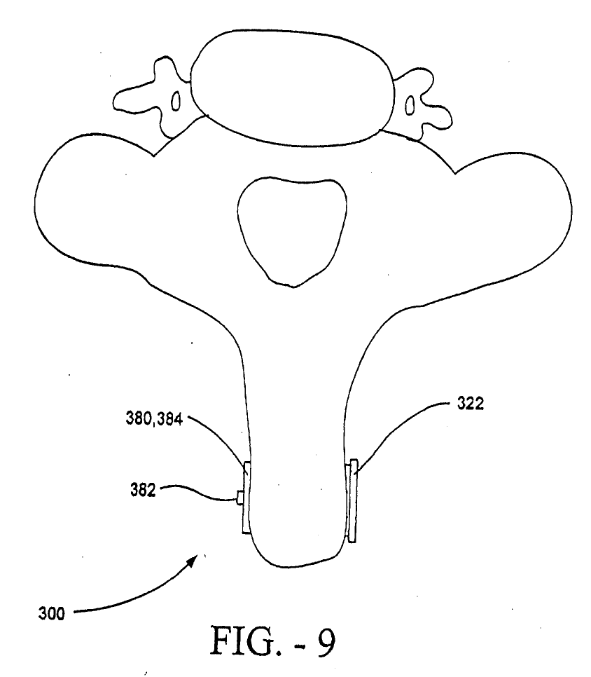

FIG. 9 depicts the embodiment of FIGS. 7 and 8 deployed between spinous processes from a top view.

A rotating tool, such as a hook mounted on the end of a rod, can be used to pull the rod 386 through the bore 319, and can be used to rotate the tissue expander 324 so that it is generally parallel to the first wing 322. In a preferred embodiment, the rod 386 can rotate the tissue expander about 90″. Alternatively, different amounts of rotation can be accomplished as needed to adapt to the anatomical structure of a patient.

Although the patient's tissue will hold the tissue expander 324 in the rotated position 242, if desired, a mechanism can be included to fix the rod 386 in the rotated position. Such mechanism can include a detent extending from head 350 which can lock into a recess in the first wing 322 as the rod 386 is pulled toward the first wing 322 and rotated when the head 350 is adjacent to the first wing 322. Another mechanism can include ridges extending from the head 350 of the rod 386 which can lock into recesses in the first wing 322.

One of ordinary skill in the art will appreciate that the locking components need not be limited to a detent and recess. The invention contemplates any locking mechanism that can secure the implant 300 in a second retention position 342 with the tissue expander 324 reconfigured to a second wing.

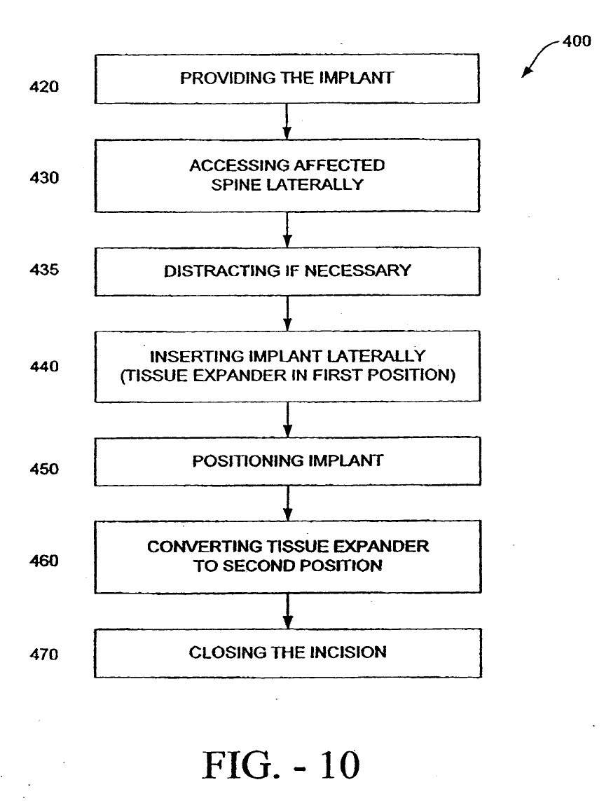

FIG. 10 depicts a method 400 of insertion of an embodiment of the invention from a lateral or postero-lateral approach. Using the disclosed method, any of implants 100, 200, and 300 can be implanted. [0044] First, an implant as in, by way of example only, embodiment 100, is provided 420, and the spine is accessed 430. Access can be accomplished laterally or posterolaterally. The implant with the tissue expander 24 in the first insertion position 36 then is inserted 440 between the spinous processes, either from the right lateral side, or the left lateral side.

The tissue expander 24 is wedge-shaped in the first insertion position 36, as described above, and is used to distract the vertebrae somewhat to facilitate the lateral insertion of the spacer 16 between the adjacent spinous processes. This level of distraction may suffice to fully insert the implant 100. However, if additional distraction is necessary prior to insertion of the tissue expander, distraction can be added prior to insertion 435, by methods already well-known in the art.

Once the implant 100 is positioned 450 with the spacer maintaining distraction of the adjacent spinous processes, the tissue expander 24 is moved from the first insertion position to the second retention position 460. For implant 100, moving the tissue expander 24 involves rotating 470 the tissue expander 24 to the second retention position 42. The rotation in one embodiment is preferably about 90″. However, varying degrees of rotation are also possible. The tissue expander 24 locks into the second retention position 42, as described above. The base element 32 in the second retention position 42 is parallel to the first wing 22, which also serves to retain the implant 100 in position and prevent displacement.

For an implant as in embodiment 200, both the first wing 222 and the tissue expander 224 are rotated together 470, because in embodiment 200, the spacer 216 is connected with the first wing 222.

For an implant as in embodiment 300, the tissue expander 324 is moved 460 from a wedge-shaped first insertion position 336 to a retaining arm or second wing second retention position 342.

After the converting step 460 the incision is closed 470.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated. It is intended that the scope of the invention be defined by the following claims and its equivalence.

What is claimed is:

1. An implant for relieving pain associated with the vertebrae of the spine and surrounding tissues and structures by maintaining and/or adding distraction between adjacent vertebrae, the spine having a sagittal plane with a lateral orientation extending across the sagittal plane, the adjacent vertebrae each having a spinous process, the implant comprising:
a rotatable spacer with a first end and second end and an axis therebetween, the spacer having an elongate cross-section that distracts the adjacent vertebrae so as to relieve the pain when implanted with the axis extending laterally between the processes;
a first wing disposed at the first end of the spacer, the first wing extending radially from the spacer; and
a tissue expander rotatably coupled to the second end of the spacer, wherein the tissue expander broadens in size from a first end to an elongate cross-section adjacent a second end, wherein the second end is adjacent to the second end of the spacer, wherein the tissue expander can rotate between a first insertion position and a second retention position, the elongate cross section of the tissue expander in the first position aligned with the elongate cross section of the spacer to facilitate axial insertion of the spacer laterally between the processes, the elongate cross section of the tissue expander in the second position extending radially from the spacer such that the tissue expander and the first wing inhibit displacement of the implanted spacer from between the processes.

2. The implant of claim 1 wherein the second retention position is maintained by at least one locking mechanism.

3. The implant of claim 2 wherein the locking mechanism locks the tissue expander into the second retention position.

4. The implant of claim 2 wherein the locking mechanism locks the first wing and the tissue expander into the second retention position.

5. The implant of claim 1 wherein the tissue expander and the first wing are rotated into the second retention position.

6. The implant of claim 1 wherein the tissue expander rotates substantially 90° from the first insertion position to the second retention position.

7. The implant of claim 1 wherein the tissue expander pivots and rotates into the second retention position.

8. The implant of claim 1 wherein the tissue expander rotates substantially 90° from the first insertion position to the second retention position, and the first wing simultaneously rotates substantially 90°.

9. The implant of claim 1 wherein the spacer has a cross-sectional shape selected from the group consisting of tear-drop, ellipse, wedge, and oval.

10. The implant of claim 1 wherein:
the first wing is substantially parallel to the sagittal plane of the spine when the implant is implanted; and
an elongated base element of the tissue expander in the first insertion position is perpendicular to the sagittal plane of the spine.

11. The implant of claim 1 wherein:
the first wing is substantially parallel to the sagittal plane of the spine when the tissue expander is in the first insertion position; and
an elongated base element of the tissue expander in the second retention position is substantially parallel to the sagittal plane of the spine.

12. An implant for relieving pain associated with the vertebrae of the spine and surrounding tissues and structures, the spine having a sagittal plane with a lateral orientation extending across the sagittal plane, the adjacent vertebrae each having a spinous process, the implant comprising:
a spacer comprising:
an elongated axis;
a first end functionally associated with a first wing, the first wing extending radially from the axis;
a second end distal to the first wing; and
an elongate cross-section that distracts the adjacent vertebrae so as to relieve the pain when implanted with the axis extending laterally between the processes
a tissue expander rotatably disposed at the second end of the spacer, the tissue expander comprising:
a first end distal to the spacer;
a base element proximal to the spacer at a second end of the tissue expander; and
a locking mechanism, wherein the tissue expander broadens in size from the first end to an elongate cross section adjacent the second end, wherein the tissue expander is adapted to rotate between a first insertion position and a second retention position, the elongate cross section of the tissue expander in the first position aligned with the elongate cross section of the spacer to facilitate axial insertion of the spacer laterally between the processes, the elongate cross section of the tissue expander in the second position extending radially from the spacer such that the tissue expander and the first wing inhibit displacement of the implanted spacer from between the processes, and the locking mechanism can engage when the tissue expander is in the second retention position.

13. The implant of claim 12 wherein:
the first wing, while the implant is being inserted, is substantially parallel to the sagittal plane of the spine; and
the base element of the tissue expander in the first insertion position is substantially perpendicular to the sagittal plane of the spine.

14. The implant of claim 12 wherein:
the first wing, when the implant has been inserted, is substantially parallel to the sagittal plane of the spine; and
the base element of the tissue expander in the second retention position is substantially parallel to the sagittal plane of the spine and to the first wing.

15. The implant of claim 12 wherein the first wing rotates together with the tissue expander as the tissue expander is rotated from the first insertion position to the second insertion position.

16. The implant of claim 15 wherein the first wing and the base element of the tissue expander in the second retention position are oriented parallel to a sagittal plane of a patient.

17. An implant for relieving pain associated with adjacent vertebrae of the spine and surrounding tissues and structures, the spine having a sagittal plane with a lateral orientation extending across the sagittal plane, the adjacent vertebrae each having a spinous process, the implant comprising:
a spacer having a first end and a second end and an axis therebetween, the spacer having an elongate cross-section that distracts the adjacent vertebrae so as to relieve the pain when implanted with the axis extending laterally between the processes;
a first wing associated with the first end of the spacer, the first wing extendable radially from the spacer and oriented substantially parallel to the sagittal plane of a patient when the implant is implanted; and
a tissue expander rotatably associated with the second end of the spacer, wherein the tissue expander broadens in size from a first end to an elongate cross section adjacent the second end, wherein the second end is adjacent to the second end of the spacer, wherein the tissue expander has a first insertion position and a second retention position rotated substantially 90° from the first insertion position, the elongate cross-section of the tissue expander in the first position aligned with the elongate cross section of the spacer to facilitate axial insertion of the spacer laterally between the processes, the tissue expander in the second retention position extending radially from the spacer along the sagittal plane of the patient and adapted to prevent displacement of the implant.

18. An implant for relieving pain associated with adjacent vertebrae of the spine and surrounding tissues and structures, the spine having a sagittal plane with a lateral orientation extending across the sagittal plane, the adjacent vertebrae each having a spinous process, the implant comprising:
a spacer having a first end and a second end and an axis therebetween, the spacer having an elongate cross-section that distracts the adjacent vertebrae so as to relieve the pain when implanted with the axis extending laterally between the processes;
a first wing associated with the first end of the spacer, the first wing extendable radially from the spacer; and
a tissue expander associated with the spacer at the second end of the spacer, wherein the tissue expander broadens in size from a first end to an elongate cross section adjacent the second end, wherein the second end is adjacent to the second end of the spacer, the tissue expander having a first insertion position and a second retention position, the broadening of the tissue expander in the first position aligned with the elongate cross section of the spacer to facilitate axial insertion of the spacer laterally between the processes, the tissue expander in the second position extending radially from the spacer such that the tissue expander and the first wing inhibit displacement of the implanted spacer from between the processes.
